(19) United States
(12) Patent Application Publication
(10) Pub. No.: US 2006/0079582 A1
(43) Pub. Date: Apr. 13, 2006

(54) Memantine for the treatment of childhood behavioral disorders

(76) Inventors: Jeffrey Jonas, Hoboken, NJ (US); Pradeep K. Banerjee, Hillsborough, NJ (US); Sandeep Gupta, Plainsboro, NJ (US); Allison Mann, Fort Lee, NJ (US); Hans-Joerg Moebius, Frankfurt (DE)

Correspondence Address: DARBY & DARBY P.C., P. O. BOX 5257, NEW YORK, NY, 10150-5257, US

(21) Appl. No.: 11/234,764

(22) Filed: Sep. 23, 2005

Related U.S. Application Data
(60) Provisional application No. 60/612,600, filed on Sep. 23, 2004.

(51) Int. Cl. A61K 31/13

(52) U.S. Cl. 514/662

(57) ABSTRACT

The present invention provides a method for the treatment of individuals diagnosed with a childhood behavioral disorder such as autistic spectrum disorders or combined type Attention-Deficit/Hyperactivity Disorder (ADHD) by administering an effective amount of memantine.

This application claims the benefit of U.S. Provisional Patent Application Ser. No. 60/612,600, filed on Sep. 23, 2004, which is herein incorporated by reference in its entirety.

### FIELD OF THE INVENTION

The present invention provides a method for the treatment of individuals diagnosed with a childhood behavioral disorder such as autistic spectrum disorders or combined type Attention-Deficit/Hyperactivity Disorder (ADHD) by administering an effective amount of memantine.

### BACKGROUND OF THE INVENTION

Childhood Disorders

The spectrum of childhood behavioral disorders include mental health problems such as anxiety disorders, Asperger's syndrome, ADHD, autistic spectrum disorders, autism, bipolar disorder, childhood disintegrative disorder, depression, disruptive behavior disorder, dyslexia, fragile X syndrome, learning disabilities, obsessive-compulsive disorder (OCD), oppositional defiant disorder, pervasive developmental disorder, reactive attachment disorder, Rett syndrome, separation anxiety disorder and Tourette's syndrome. These childhood disorders interfere with a child's normal development and functioning. In the U.S., 1 in 10 children and adolescents suffer from mental illness severe enough to cause some level of impairment. In any given year, however, it is estimated that fewer than 1 in 5 of these children receives needed treatment.

Autistic spectrum disorders and ADHD are two of the most commonly diagnosed neurobehavioral disorders of children. Autistic spectrum disorders are considered brain disorders that typically affect an individual's ability to communicate, form relationships with others, and respond appropriately to the environment. They are characterized by qualitative impairments in three core sets of symptoms: social interaction, communication, and behavior and play (which is often restrictive, repetitive, and/or stereotyped in nature) (Tidmarch et al., Can. J. Psychiatry 2003; 48: 517-525). ADHD is a clinically heterogeneous disorder characterized by varying degrees of inattentiveness, and/or hyperactivity-impulsivity. In some cases ADHD has a familial component. The three major clinical types, as defined in the Diagnostic and Statistical Manual of Mental Disorders, 4th edition text revision (DSM-IV-TR™—American Psychiatric Association (2000), Diagnostic and Statistical Manual of Mental Disorders, 4th edition text revision. Washington, D.C.: American Psychiatric Association), are the i) predominantly inattentive type; ii) predominantly hyperactive-impulsive type; and iii) combined type (with significant symptoms of both inattentiveness and hyperactivity-impulsivity). The combined type is the most common subgroup (50% to 75%), followed by the inattentive type (20% to 30%) and the hyperactive-impulsive type (less than 15%) (Wilens et al., Annu. Rev. Med. 2002; 53: 113-131). Symptoms can often interfere with academic, home, or social functioning. It has been reported that about 30-50% of childhood ADHD persists into adulthood. (Barberesi et al., Arch. Pediatr. Adolesc. Med. 2002; 156: 217-224; Kordon et al., Psychother. Psychosom. Med. Psychol. 2004 March; 54(3-4):124-36).

Autistic Spectrum Disorder

Autistic spectrum disorders include autism, Asperger's syndrome, pervasive development disorder and childhood disintegrative disorder. Recent studies estimate the prevalence of autism, the most common of autistic disorders, occurs within a range of about 2.5/10,000 to 30.8/10,000 with a median estimate being 10/10,000. Although the reasons are unclear, there is evidence that prevalence of autism is increasing over time; it is 3 to 4 times higher than 30 years ago (Fombonne, JAMA 2003; 289: 87-89). Some individuals with autism are relatively high-functioning, with speech and intelligence intact. Others are mentally retarded, mute, or have serious language delays. For some, autism makes them seem closed off and shut down; others seem locked into repetitive behaviors and rigid patterns of thinking.

Autistic spectrum disorders becomes evident from infancy to the first 3 years of age, when the child fails to develop typical patterns and behaviors of normal child development associated with social development, language skills, and sensory symptoms. Children diagnosed with autistic spectrum disorders demonstrate poor or limited social relationships, underdeveloped communication skills, exhibit repetitive behaviors, interests, and activities, and demand consistency in their environment. Some are painfully hypersensitive to sound, touch, sight, or smell. Autistic spectrum disorders can also be associated with self-injurious behavior, such as head banging, hair pulling, and biting.

It is generally believed that autistic spectrum disorders are caused by abnormal brain development, during pregnancy and early childhood development. Evidence suggests that deficits in social cognition and communication in autism may be related to dysfunction in the amygdala, hippocampus, and related limbic and cortical structures (Tuchman, Neurol. Clin. 2003; 21(4):915-32, viii), as well as the cerebellum and brainstem (Keller et al., Mol. Neurobiol. 2003; 28(1):1-22.

Although autistic spectrum disorders appear to be hereditary, specifics and extent of any inheritable phenotype remains unknown. Although recent data suggest that there are autism susceptibility genes, none have been directly linked to causation (Buxbaum et al., Mol. Psychiatry. Mol. Psychiatry. 2004; 9(2):144-50; Bacchelli et al, Mol. Psychiatry. 2003; 8(11):916-24).

Defects in neurotransmitter systems, notably the serotonergic, but also the cholinergic and GABAergic systems, are also being investigated as playing a role in autistic spectrum disorders. Within the past few years, clinical observations by Horvath et al. hypothesized that a defect in secretin and its receptors may play a role in autism (J. Assoc. Acad. Minor. Physicians 1998; 9: 9). However, a synthetic form of the natural human hormone secretin developed by RepliGen for the potential treatment of autism did not demonstrate significant improvement in autistic behavior compared to placebo (Sponheim, Acta. Paediatr. 2002; 91(5):540-5).

There are no existing pharmacologic treatments that have been successful in treating the core symptoms of autism, namely the profound social impairment and inability to communicate. Currently, there are no Food and Drug Administration-approved drugs specifically indicated for the treatment of autism or the core symptoms of autism. Current drug therapies focus on those that are active on the dopaminergic, serotonergic, and opiate systems (Baghdadli et al., Encephale. 2002; 28: 248-540). A number of drug classes (including antipsychotics, antidepressants, and antiepileptics) have been used in autistic patients with variable effect. The newer antipsychotics, psychostimulants, presynaptic noradrenergic blocking agents (clonidine and guanfacine) and selective serotonin reuptake inhibitors (SSRIs) were shown to reduce impairing complicating symptoms of affective instability, irritability, hyperactivity and inattentiveness, aggression, self-injury and stereotypies. For example, stimulants such as methylphenidate (Ritalin®) and amphetamines are often prescribed for young children, while typical and atypical anti-psychotics are used for adults.

For disruptive/irritable and self-injurious behavior, lithium, fenfluoramine, naltrexone and other opiate blockers, serotonin reuptake inhibitors, beta-blockers, haloperidol and risperidone have been employed. For repetitive motor activities and stereotyped motions, drugs typically used to treat obsessive-compulsive disorders are prescribed. These include SSRIs and clomipramine, typical antipsychotics, and risperidone. For anxiety associated with autism, SSRIs such as fluvoxamine and buspirone are typically prescribed, with benzodiazepines as potentially helpful in patients with concomitant mental retardation. For a brief review see Lindsay et al., Pediatr. Ann. 2003; 32(10): 671-6 and Stigler et al., Exp. Rev. Neurotherapeutics 2002; 2(4): 499-510).

Despite progress reported for the treatment of autistic spectrum disorders with SSRIs such as fluvoxamine, fluoxetine, and sertraline for targeted behaviors such as perseverative behavior, currently prescribed pharmaceuticals (e.g., SSRIs, and the above-listed drugs) fail to benefit the primary symptoms and can have marked adverse effects. Accompanying adverse effects include restlessness, hyperactivity, agitation, insomnia and decreased appetite. Similarly, these medications are typically effective in high doses from about 20-80 mg/day, which could become intolerable due to side effects. For example, risperidone treatment was associated with weight gain, increased appetite, fatigue, drowsiness, tremor and drooling.

The medication-refractory status of the social and communicative deficits associated with autistic spectrum disorders is likely due to the as yet unidentified neurochemical basis of autistic spectrum disorders, and the lack of involvement of the neurotransmitter systems (dopamine, noradrenaline and serotonin) in the pathophysiology of social and communicative behavior (Buitelaar, Novartis Found. Symp. 2003; 251:235-44.

Despite the foregoing, recent research demonstrating possible defective frontal lobe defects in cholinergic receptors in autistic individuals has led to attempts at employing cholinesterase inhibitors such as donepezil for autism (Perry et al., Am. J. Psychiatry. 2001; 158(7):1058-661; and Hardan et al., J. Child. Adolesc. Psychopharmacol. 2002; 12(3): 237-4). Other cholinesterase inhibitors include galantamine and rivastagmine. The destruction of cholinergic neurons has also been attributed to excess activation of glutamate receptors, particularly the NMDA subtype (Li et al., J. Neuropathol. Exp. Neurol. 1997; 56(8):901-11).

In autism, glutamate levels may be increased and glutamate receptors up-regulated as part of an excitotoxic process that damages neural networks, damage that may in turn contribute to some of the core symptoms of autism. Alterations of the levels of Bcl-2 and P-53 (proteins that are markers of apoptosis) in the frontal, parietal, and cerebellar cortices (Araghi-Niknam et al., Cell. Mol. Neurolol. 2003; 23: 945-52) as well a decrease in the cerebellar protein Reelin, a glycoprotein responsible cell layering, in the brains of autistic subjects suggest that apoptotic mechanisms may play a role in the disease process. In addition, there is evidence that excitatory amino acids are increased in those with autism. Glutamate levels are increased in the blood and platelets of autistic subjects (Aldred et al., J. Autism Dev. Disord. 2003; 33: 93-97; Moreno-Fuemnayor et al., Invest. Clin. 1996; 37: 113-128). Glutamic acid decarboxylase, an enzyme responsible for the conversion of glutamate to gamma amino butyric acid (GABA), is deficient in the autistic parietal and cerebellar cortex and could lead to an excess of glutamate in those brain regions. Increases in glutamate have also been identified in the cerebrospinal fluid of patients with Rett disorder, a disorder classified as part of the autistic spectrum (Riikonen, J. Child. Neurol. 2003; 18: 693-697).

Attention-Deficit/Hyperactivity Disorder

An imbalance of catecholamine neurotransmitters has been implicated in the pathophysiology of Attention-Deficit/Hyperactivity Disorder (ADHD). Dopamine, in particular, is believed to play a major role in ADHD. Findings include hypoactivity of catecholamine-rich fronto-subcortical systems, elevations in striatal dopamine transporter (DAT) levels (Dougherty et al., Lancet 1999; 354: 2132-2133; Cheon et al., Eur. J. Nucl. Med. Mol. Imaging 2003; 30: 306-311), an association between ADHD and the DAT gene (Cook et al., Am. J. Med. Gen. 1995; 56: 993-998), and an association between ADHD and the D4 receptor gene (DRD4) (Faraone et al., Am. J. Psychiatry 1999; 156: 768-770; Benjamin et al., Nat. Genet. 1996; 12: 81-84).

All current U.S. drugs labeled for use in ADHD are directed at modulating CNS catecholamine levels. With one exception, (atomoxetine HCl, trade name: Straterra®), they belong to the stimulant drug class (i.e., methylphenidate, pemoline, and dextroamphetamine). Stimulants increase synaptic levels of dopamine (and norepinephrine as well as serotonin) by blocking the presynaptic monoamine reuptake in neurons. About 30% of ADHD patients fail to respond to any given stimulant drug and there is no clear evidence of a differential response to different stimulants (Wilens et al., J. Am. Acad. Child. Adolesc. Psychiatry 1996; 35: 409-432). Despite recent studies which suggest that treatment with stimulants may reduce the risk of future substance abuse in ADHD patients (Biederman et al., J. Clin. Psychiatry. 2003; 64 Suppl 11:3-8), stimulants are considered drugs with abuse potential and as such are classified as controlled substances and scheduled by the DEA under Federal law (e.g., methylphenidate, dextroamphetamine—Schedule II [high abuse potential]; pemoline—Schedule IV [low abuse potential]). Thus, the development of additional safe and effective, non-stimulant pharmacologic options would be of great clinical benefit to the ADHD population.

Several lines of research suggest that glutamate and NMDA receptor activity may play a role in the pathophysiology of ADHD. Levels of glutamate in the prefrontal cortex and striatum of pediatric patients with ADHD are elevated, and return to normal following drug treatment (Carey et al., Clin. Neuropharmacol. 2003; 26: 218-221). In a genetic rat model of ADHD, the spontaneously hypertensive rat (SHR), prefrontal cortex has shown increased functional activity of AMPA subtype of glutamate receptors (Russell et al., Metab. Brain Dis. 2001; 16: 143-149). Although NMDA receptor activity has not been studied in these rats, there is evidence that enhanced AMPA receptor function increases NMDA receptor activity. In the hippocampal CA1 region, activation of dopamine D4 receptors can selectively decrease NMDA receptor function via activation of the platelet-derived growth factor β (PDGF-β) receptors. Since the regional distribution of NMDA receptors and dopamine D4 receptors in the limbic and cortical brain regions are similar, it is quite possible that a bidirectional, reciprocal signaling relationship exists. That is, NMDA receptor antagonism is likely to mediate an increase in dopamine D4 receptor-mediated signaling.

U.S. Pat. No. 4,994,467 describes a method for treating autism in children by administration of an effective amount of a N-methyl-D-aspartate receptor antagonist selected from the group consisting of ketamine and dextromethorphan. U.S. Pat. No. 6,362,226 describes a method of treating autism in a patient comprising administering an N-methyl-D-aspartate receptor antagonist. More specifically, the '226 patent specifies the treatment wherein the N-methyl-D-aspartate receptor antagonist comprises dextromethorphan. Neither the '226 nor the '467 patent describes the use of 1-amino-alkylcyclohexanes (i.e., memantine) for the treatment of autism. U.S. Pat. No. 5,614,560 describes a method for reducing non-ischemic NMDA receptor-mediated neuronal degeneration using aminoadamantanes, including memantine. The '560 does not describe the treatment of autistic spectrum disorders or ADHD. Lastly, published application WO 03/061656 describes a method of treating CNS disorders using a combination therapy of a GABA analog and a non-toxic NMDA receptor antagonist, including memantine. The '656 application does not describe the use of memantine as a monotherapy.

Memantine

It has been shown that 1-aminoadamantane (amantadine), produced statistically significant improvement in measures of hyperactivity and irritability in a small, double-blind, placebo-controlled study in autistic children (King et al. J. Am. Acad. Child. Adolesc. Psychiatry 2001; 40; 658-65). In view of recent research that links autism to defects in the cholinergic pathway, described above, the present inventors hypothesized that memantine, which is a NMDA glutamate receptor antagonist effective for the treatment of Alzheimer's disease (as well as Parkinson's and other neurological diseases), may be effective for the treatment of autism, ADHD and other autistic spectrum disorders.

Memantine (1-amino-3,5-dimethyl adamantane), which is disclosed, e.g., in U.S. Pat. Nos. 4,122,193; 4,273,774; and 5,061,703, is a systemically-active uncompetitive NMDA receptor antagonist having low to moderate affinity for the receptor and strong voltage dependency and rapid blocking/unblocking kinetics. Memantine hydrochloride is currently available in the U.S. and in over 42 countries worldwide. It is approved for the treatment of moderate to severe Alzheimer's disease (AD) in the United States at a dose of up to 20 mg/day (5-10 mg BID). The approval of memantine (trade name: Namenda®) was based on the results of 2 randomized, double-blind, placebo-controlled clinical studies performed in the United States. These studies, performed using outpatients ages 50-93 with moderate to severe dementia of the Alzheimer's type (MMSE 5-14 and 3-14, respectively), demonstrated significant improvement in cognitive, functional, and global assessments (Tariot et al., JAMA 2004; 291: 317-324; Reisberg et al., N. Engl. J. Med. 2003; 348: 1333-1341). A third randomized, double-blind, placebo-controlled study (9403) was performed in Latvian nursing home patients with moderately severe to severe Alzheimer's disease or vascular dementia (MMSE<10), and demonstrated a significant improvement in care dependency and global change for memantine relative to placebo (Winblad et al., Int. J. Geriatr. Psychiatry 1999; 14: 135-46).

Pharmacokinetic studies in adult humans have demonstrated that memantine is 100% bioavailable after an oral dose, undergoes minimal metabolism, and exhibits a terminal elimination half-life of 60 to 80 hours (57%-82% or more of the dose is eliminated intact in the urine). It rapidly crosses the blood brain barrier with a CSF/serum ratio of 0.52. Memantine does not inhibit cytochrome P-450 (CYP 450) isoenzymes in vitro, so that no pharmacokinetic interactions with drugs metabolized by these enzymes are expected. Its pharmacokinetic profile is not affected by food, sex, or age. Renal clearance involves active tubular secretion moderated by pH dependent tubular reabsorption.

Memantine has exhibited an acceptable safety and tolerability profile in 2297 patients in 27 clinical trials involving a variety of neurodegenerative disorders (e.g., dementia, neuropathic pain, spasticity, and Parkinson's disease). The most common adverse events observed in clinical trials have been dizziness, headache, constipation, and confusion, but even these are relatively rare.

No published clinical studies in the autism or ADHD populations have been performed with memantine. However, the uncompetitive NMDA receptor antagonist amantadine has demonstrated activity in children with other behavioral disturbances. In a small double-blind, placebo-controlled study in autistic children, amantadine produced statistically significant improvement in measures of hyperactivity and irritability. There are additional reports of favorable effects in hyperkinetic children (Mattes, Psychopharmacol. Bull. 1980; 16: 67-69), and children with attention deficit disorder (ADD) (Masters et al., J. Am. Acad. Child. Adolesc. Psychiatry 1997; 36: 301).

Therefore, based on clinical experience and experimental evidence, it is hypothesized that the NMDA receptor antagonist memantine will be an effective treatment for both autistic spectrum disorders and combined type ADHD. By decreasing the NMDA receptor activity with memantine the D4 receptor signaling/activity could be modified and lead to salutary effects in ADHD. However, according to DSM-IV-TR, combined type ADHD differs from both hyperactivity (predominantly hyperactive) and ADD (predominantly inattentive), based on a predominance of symptoms of inattention, or a predominance of symptoms of hyperactivity-impulsivity. Furthermore, in view of the fact that conventional antipsychotic medication and SSRIs commonly prescribed to pediatric patients with autistic spectrum disorder require doses similar to those prescribed in adults, resulting in a high incidence of severe adverse reactions, there is a need in the art for drug therapies that can be administered at lower doses or that present fewer adverse effects.

### BRIEF DESCRIPTION OF THE DRAWINGS

FIG. 1. FIG. 1 demonstrates the distribution of scores in an LOCF analysis in the ITT population for cohort 1 at baseline, week 4, and week 8.

FIG. 2. FIG. 2 demonstrates the distribution of scores in an LOCF analysis in the ITT population for cohort 2 at baseline, week 4, and week 8.

FIG. 3. FIG. 3 demonstrates the distribution of scores in an OC analysis in the ITT population for cohort 1 at baseline, week 4, and week 8.

FIG. 4. FIG. 4 demonstrates the distribution of scores in an OC analysis in the ITT population for cohort 2 at baseline, week 4, and week 8.

### SUMMARY OF THE INVENTION

The present invention provides a method for the treatment of autistic spectrum disorders or combined type ADHD by administering an effective amount of memantine to a subject in need thereof.

In one embodiment, the subject is a child between the ages of about 5 and about 17.

In another embodiment, memantine is administered in a range from about 1.25-100 mg/day.

In one specific embodiment, the memantine is administered at about 5-20 mg/day.

In another specific embodiment, memantine is administered at about 10-20 mg/day.

In further specific embodiment, memantine is administered at about 20 mg/day.

In another embodiment, memantine is administered in a flavored, oral, liquid formulation.

In another embodiment, memantine is administered in a modified release formulation.

In another embodiment, the subject in need thereof is an adult. In this embodiment, memantine is administered at a dosage in a range from about 5-100 mg/day. In a specific embodiment, memantine is administered at a dosage in a range from about 20-40 mg/day.

### DETAILED DESCRIPTION

The present invention provides a method for the treatment of autistic spectrum disorders or combined type ADHD, according to the criteria set forth in DSM-IV-TR®, in pediatric patients (i.e. up to about an age of 17) by administering an effective amount of memantine.

It is hypothesized that both autistic spectrum disorders and ADHD may involve a derangement in glutamatergic brain activity which may contribute to some of the disorder's clinical manifestations. It is further hypothesized that, by modifying glutamatergic activity, the uncompetitive NMDA receptor antagonist memantine could provide clinical benefit to people with autistic spectrum disorders or combined type ADHD. Accordingly, clinical and experimental evidence regarding the safety and efficacy will be generated according to open label studies as described further below. Duration of treatment may be as short or as long as necessary, and may extend into adulthood.

### Definitions

“Memantine” refers to 1-amino-3,5-dimethyladamantane hydrochloride. In the United States, the trade name for memantine is Namenda®, in Germany it is Akatinol® and Auxura®, and it is Ebixa® in the remainder of the European Union. Memantine is the subject matter of U.S. Pat. Nos. 4,122,193, 4,273,774 and 5,061,703.

Various salts of memantine can be used. The term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydriodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., memantine) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. However, since memantine is highly soluble, aqueous solutions are preferred. Suitable pharmaceutical carriers are described in “Remington's Pharmaceutical Sciences” by E. W. Martin, 18^{th} Edition. Particularly preferred for the present invention are carriers suitable for immediate-release, i.e., release of most or all of the active ingredient over a short period of time, such as 60 minutes or less, and make rapid absorption of the drug possible.

The term “subject in need thereof” as used herein refers to a mammal. In particular, the term refers to humans diagnosed with an autistic spectrum disorder or combined type ADHD.

The term “autistic spectrum disorder” refers to autism-related disorders which include autism, Asperger's syndrome, pervasive developmental disorder (PDD), Rett disorder and childhood disintegrative disorder. Such disorders can be diagnosed using the DSM-IV or ICD-10 criteria or other diagnostic tools such as the Checklist for Autism in Toddlers (CHAT), Childhood Autism Rating Scale (CARS), Parent Interviews for Autism (PIA), Gilliam Autism Rating Scale (GARS), Behavior Rating Instrument for Autistic and other Atypical Children (BRIAC), Autism Diagnostic Interview-Revised (ADI-R), Autism Diagnostic Observation Schedule-Generic (ADOS-G), and Diagnostic Interview for Social and Communication Disorders (DISCO).

According to DSV-IV-TR™, a diagnosis of autism is made when the patient demonstrates a total of six (or more) items from (1), (2), and (3), with at least two from (1), and one each from (2) and (3):
(1) qualitative impairment in social interaction, as manifested by at least two of the following:
   (a) marked impairment in the use of multiple nonverbal behaviors such as eye-to-eye gaze, facial expression, body postures, and gestures to regulate social interaction;
   (b) failure to develop peer relationships appropriate to developmental level;
   (c) a lack of spontaneous seeking to share enjoyment, interests, or achievements with other people (e.g., by a lack of showing, bringing, or pointing out objects of interest);
   (d) lack of social or emotional reciprocity;
(2) qualitative impairments in communication as manifested by at least one of the following:
   (a) delay in, or total lack of, the development of spoken language (not accompanied by an attempt to compensate through alternative modes of communication such as gesture or mime);
   (b) in individuals with adequate speech, marked impairment in the ability to initiate or sustain a conversation with others;
   (c) stereotyped and repetitive use of language or idiosyncratic language;
   (d) lack of varied, spontaneous make-believe play or social imitative play appropriate to developmental level;
(3) restricted repetitive and stereotyped patterns of behavior, interests, and activities, as manifested by at least on of the following:
   (a) encompassing preoccupation with one or more stereotyped and restricted patterns of interest that is abnormal either in intensity or focus;
   (b) apparently inflexible adherence to specific, nonfunctional routines or rituals;
   (c) stereotyped and repetitive motor mannerisms (e.g., hand or finger flapping or twisting, or complex whole-body movements);
   (d) persistent preoccupation with parts of objects.

In addition, delays or abnormal functioning in at least one of the following areas, with onset prior to age 3 years: (1) social interaction, (2) language as used in social communication, or (3) symbolic or imaginative play. Lastly, care should be taken to ensure that the disturbance is not better accounted for by Rett Disorder or Childhood Disintegrative Disorder.

In accordance with the present invention, the following instruments may be used to diagnose and/or evaluate efficacy of memantine for the treatment of autistic spectrum disorders.

The Autistic Diagnostic Observation Schedule (ADOS-G). The ADOS-G is a standardized observation of social and communicative behavior performed directly with the child over 20-40 minutes (Lord et al., J. Autism Dev. Disord. 1989; 19(2): 185-212). It is organized in four overlapping modules according to the expressive language level of the subject.

Test of Nonverbal Intelligence, Third Edition (TONI-3). The TONI-3 is a reliable and validated measure of intelligence, aptitude, abstract reasoning, and problem-solving skills without relying on verbal ability or English-language proficiency (Brown et al., Test of Non-Verbal Intelligence (3rd. ed.). Austin, Tex.: PRO-ED). It contains 50 items arranged in easy to difficult order. Raw scores are converted to percentile ranks and to deviation quotients with a mean of 100 and a standard deviation of 15 points. To maintain the language-free format, instructions are pantomimed and responses are conveyed by pointing or making some other meaningful gesture. It takes approximately 15 minutes to administer and is appropriate for persons 5 through 85 years.

Clinical Global Impression-Improvement (CGI-I). This is a 7-point Likert scale which is anchored at a score of 4 (no change), with a score of 1 corresponding to a rating of “very much improved” and a score of 7 corresponding to a rating of “very much worse.” It will be assessed at Visits 3 to 10, or upon early study discontinuation

Clinical Global Impression—ADHD-Severity (CGI-S). This is a 7-point scale (1 [not ill] to 7 [extremely ill]) to be assessed by the Investigator at Screening, Baseline, and Visits 3 to 10, or upon early study discontinuation.

Research Unit Psychopharmacology Autism Network Target Symptoms Assessment (RUPP-TAS). In this assessment, behaviors (usually 1 or 2) of concern to the caregiver are quantified are collected by blinded physicians (who may do CGI assessments) but rated for outcome by other blinded (third-party) clinical judges on a 9-point scale of change from baseline. Information is elicited using a semi-structured 5 to 10 minute interview of the caregiver bar. The test takes approximately 14 minutes to complete. It will be administered at Baseline (end of week 0), Visit 6 (end of week 4), and Visit 10 (end of week 8) or upon early discontinuation (Arnold et al., Psychiatry 2003; 42: 1143-1450).

Aberrant Behavior Checklist—Community Version (ABC-CV). The ABC-CV is a symptom checklist for assessing problem behaviors of children and adults with mental retardation (Aman et al., Am J Mental Deficiency 1985; 89: 485-491). There are 58 items resolve into five subscales: (1) Irritability, Agitation, (2) Lethargy, Social Withdrawal, (3) Stereotypic Behavior, (4) Hyperactivity, Noncompliance, and (5) Inappropriate Speech. The checklist is completed by the caregiver at Baseline (end of week 0), Visit 6 (end of week 4) and Visit 10 (end of week 8) or upon early study discontinuation.

Peabody Picture Vocabulary Test III (PPVT III). The PPVT III is a reliable and validated measure of receptive vocabulary for standard English and a screening test of verbal ability for ages 2½ and older (Dunn et al., Peabody Picture Vocabulary Test—Third Edition (PPVT-III). 1997, Circle Pines, Minn.: AGS Publishing). Each form contains four training items followed by 204 test items divided into 17 sets of 12 items each. Each item has four simple, black-and-white illustrations on a Picture Plate or page arranged in a multiple-choice format. The test taker selects the picture considered to best illustrate the meaning of a stimulus word presented orally by the examiner. The test takes 11 to 12 minutes to administer. It will be administered at Baseline (end of week 0), Visit 6 (end of week 4), and Visit 10 (end of week 8) or upon early discontinuation.

Matching to Sample (MTS) task. This is a computerized color memory test in which the child is shown a color (Aman et al., CNS Spectrums 2004; 9: 36-47). The child presses the screen where the stimulus appears; three colors appear with a delay. The duration of delay is adjusted depending on the test takers accuracy and overall ability. It will be administered at Baseline (end of week 0), Visit 6 (end of week 4), and Visit 10 (end of week 8) or upon early discontinuation. The term “combined type ADHD” refers to a diagnosis of six or more symptoms of inattention, and six or more symptoms of hyperactivity-impulsivity (according to DSM-IV-TR™) that have persisted for at least six months.

The Kiddie Schedule for Affective Disorders and Schizophrenia—Present and Lifetime (K-SADS-PL) is a semi-structured diagnostic interview that assesses the major diagnostic criteria relevant to psychiatric disorders in pediatric patients and adolescents, including ADHD (Kaufman et al., Version 1.0 of October 1996 ed. Pittsburgh, Pa.: Dept. of Psychiatry, Pittsburgh School of Medicine). It evaluates both past and current episodes and will be used in this study to establish that the patient meets DSM-IV-TR™ criteria for ADHD-combined type and to rule out other psychiatric diagnoses. The K-SADS-PL is composed of 6 sections: 1) an unstructured introductory interview; 2) a Diagnostic Screening Interview; 3) the Supplement Completion Checklist; 4) the appropriate Diagnostic Supplements; 5) the Summary Lifetime Diagnoses Checklist; and 6) the Children's Global Assessment Scale (C-GAS) ratings. The unstructured introductory interview takes approximately 10-15 minutes to complete. The duration of the remainder of the interview will vary from patient to patient.

The Peabody Picture Vocabulary Test III (PP VT III) can identify patients, with a standardized score of ≧70, considered to be in the non-mentally retarded range (a criterion for ADHD diagnosis).

The Attention Deficit/Hyperactivity Disorder Rating Scale, Fourth Edition —Parent Version (ADHD-IV-RS) assesses each of the 18 individual criteria symptoms of ADHD in DSM-IV-TR™ on a severity grid (0=not present, 3=severe; overall minimum score=0, maximum score=54) (DuPaul et al., ADHD Rating Scale-IV: Checklists, Norms, and Clinical Interpretations 1998. New York: The Guilford Press). Inattention symptoms comprise the odd-numbered items, and hyperactive-impulsive symptoms are represented by the even-numbered items. The scale takes approximately 10 to 15 minutes to complete. This is a valid and reliable scale that has been shown to be sensitive to drug effects in pediatric and adult groups. It is to be completed by the patient's parent or legal guardian at Screening (part 1.1), Baseline, and from two to eight times during Visits 3 to 10, or upon study discontinuation.

The Clinical Global Impression-ADHD-Severity (CGI-S) is a 7-point scale (1 [not ill] to 7 [extremely ill]) to be assessed by the Investigator at Screening (part 1.1), Baseline, and from two to eight times during Visits 3 to 10, or upon study discontinuation.

Conner's Continuous Performance Test II is a computerized test which assesses sustained attention and freedom from distractibility. A single letter is presented in the middle of a computer screen (Connors, Connors'Continuous Performance Test II Computer Program for Windows. North Tonawanda, N.Y.: Multi-Health Systems). If the letter is an X, the patient is instructed not to press the space bar. If it is any letter other than an X the patient is asked to press the space bar. The test takes approximately 14 minutes to complete. It will be administered at Baseline and Visit 10 or upon study discontinuation.

The Woodcock Johnson III (WJ III)—math fluency test is one of a battery of tests from the Woodcock Johnson III Tests of Achievement. It assesses the speed of performing simple calculations in 3 minutes and will be administered at Baseline and Visit 10 or upon study discontinuation.

The Woodcock Johnson III (WJ III)—reading fluency test is one of a battery of tests from the Woodcock Johnson III Tests of Achievement. It takes approximately 3 minutes to complete. It assesses reading speed and will be administered at Baseline and Visit 10 or upon study discontinuation (Woodcock et al., Woodcock Johnson III Tests of Achievement 2000; Riverside Publishing).

The Stroop Test is a test of selective attention which examines the relationship between color naming and word reading. It takes approximately 5 minutes to complete. It will be administered at Baseline and Visit 10 or upon study discontinuation (Golden et al., STROOP Color and Word Test Children's Version for Ages 5-14 2003: Illinois, Stoeting Co.).

The term “treat” is used herein to mean to relieve or alleviate at least one symptom of a disease in a subject. For example in relation to autism, this includes symptoms in relation to the development of language skills, improvements in eye contact, improved ability to function socially and/or tolerate mild changes in routine, improved response to verbal commands, improved awareness of surroundings, decreased aggression, or self-injurious, hyperactive, or repetitive behavior, and improved sensory perception. For example, in relation to combined type ADHD, this includes symptoms of both inattentiveness and hyperactivity-impulsivity, improvement of attention to detail, reduction of careless mistakes, improved attention to tasks and play activities, improved listening when spoken to directly, improvements in follow-through with instructions and schoolwork, improvements in organizing tasks and activities, improved engagement in tasks that require sustained mental effort, reduction in loss of objects necessary for daily activity, reduction in distraction by extraneous stimuli, reduction in forgetfulness in daily activities, reduction in fidgeting or squirming, improvement in remaining seated when required or expected, reduction in excessive running or climbing in inappropriate situations, reduction in restlessness or behavior described as “driven like a motor”, improvement in engaging in quiet leisure activity, reduction in excessive talking or chattering, reduction in inappropriate blurting out of answers or interrupting of others conversations or activities, and/or improvement in social, academic or occupational functioning.

Within the meaning of the present invention, the term “treat” also denotes to arrest, delay the onset (i.e., the period prior to clinical manifestation of a disease) and/or reduce the risk of developing or worsening a disease, such as a childhood behavioral disorder such as autism or ADHD. For example, it is known that there is a familial pattern of ADHD, whereby first-degree biological relatives of children with ADHD is more common than in the general population.

The term “therapeutically effective amount” is used herein to mean an amount or dose of memantine that is effective to ameliorate, delay, or prevent any of the foregoing symptoms, behaviors or events associated with autistic spectrum disorders or combined type ADHD.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a give value. Numerical quantities given herein are approximate unless stated otherwise, meaning that the term “about” or “approximately” can be inferred when not expressly stated.

### Formulation, Dosage, and Administration

In conjunction with the methods of the present invention, also provided are pharmaceutical compositions comprising a therapeutically effective amount of memantine. The compositions of the invention further can comprise a carrier or excipient (all pharmaceutically acceptable). The compositions can be formulated for once-a-day administration or twice-a-day administration.

Memantine (NAMENDA™) is commercially available as the hydrochloride salt in 5 or 10 mg film-coated tablets. However, according to the present invention, the dosage form of memantine may be a solid, semisolid or liquid formulation according to the following.

Memantine may be administered orally, topically, parenterally, or mucosally (e.g., buccally, by inhalation, or rectally) in dosage unit formulations containing conventional non-toxic pharmaceutically acceptable carriers. It is usually desirable to use the oral route. In a preferred embodiment for the administration to pediatric subjects, memantine is formulated as a flavored liquid, e.g., peppermint flavor. Memantine may be administered orally in the form of a capsule, a tablet, or the like, or as a semi-solid or liquid formulation (see Remington's Pharmaceutical Sciences, Mack 5 Publishing Co., Easton, Pa.). The orally administered medicaments may also be administered in the form of a time-controlled release vehicle, including but not limited to diffusion-controlled systems, osmotic devices, dissolution-controlled matrices, and erodible/degradable matrices.

For oral administration in the form of a tablet or capsule, memantine can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets can be coated by methods well known in the art. The cores may also be coated with a concentrated sugar solution which may contain e.g., gum arabic, gelatine, talcum, titanium dioxide, and the like. Alternatively, the tablets can be coated with a polymer known to a person skilled in the art, wherein the polymer is dissolved in a readily volatile organic solvent or mixture of organic solvents. In preferred embodiments, memantine is formulated in to immediate-release (IR) or modified-release (MR) tablets. Immediate release solid dosage forms permit the release of most or all of the active ingredient over a short period of time, such as 60 minutes or less, and make rapid absorption of the drug possible. Modified release solid oral dosage forms permit the sustained release of the active ingredient over an extended period of time in an effort to maintain therapeutically effective plasma levels over similarly extended time intervals and/or to modify other pharmacokinetic properties of the active ingredient.

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The compositions of the invention can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers useful in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

In one embodiment, the compositions of the invention are formulated into immediate-release or modified release beads in an oral dosage form. Beads offer advantages over conventional solid oral modified release dosage forms, such as tablets. Beads are dose proportional, i.e., the same proportions of beads of different types can be used for different doses without significantly altering the percent drug released over time. Different doses are obtained by using different amounts of beads. Beads also enable a variety of dissolution profiles by mixing one or more types of beads with different dissolution properties or using multi-layer coatings, as additional drug layering over a polymer layer and subsequent coatings to prepare unitary beads, as familiar to one skilled in the art. Such dissolution profiles may or may not be possible using modified release tablet formulations. Beads also enable a wide range of drug loading. For example, memantine beads may be loaded on beads at up to 500 mg/g. Memantine bead formations are described in detail in commonly-owned patent application Ser. No. 60/691,512 filed on Jun. 16, 2005, which is herein incorporated by reference in its entirety.

Formulation of memantine in semi-solid or liquid form is within the skill of the art, as the active ingredient is highly soluble in aqueous media. Usually the active substance, i.e., memantine, will constitute between 0.1 and 99% by weight of the formulation, more specifically between 0.5 and 20% by weight for formulations intended for injection and between 0.2 and 50% by weight for formulations suitable for oral administration.

In a preferred embodiment of the inventions, memantine is administered in a modified release formulation. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks. In light of the advantages of modified release dosage forms, it has been the objective of many skilled in the art to develop such dosage forms.

The majority of modified release dosage forms comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In a specific embodiment of the invention, memantine is formulated in an oral, liquid formulation. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. A particular example of an oral time-controlled release pharmaceutical formulation is described in U.S. Pat. No. 5,366,738.

For oral administration in liquid form, memantine can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of memantine, with the balance being sugar and mixture of ethanol, water, glycerol and propylene glycol. Optionally such liquid formulations may contain coloring agents, flavoring agents, saccharine and carboxymethyl-cellulose as a thickening agent or other excipients known to a person skilled in the art.

In one specific embodiment, a therapeutically effective amount of memantine is administered in an oral solution containing a preservative, a sweetener, a solubilizer, and a solvent. The present oral solution may include one or more buffers, flavorings, or additional excipients. In a further preferred embodiment, a peppermint or other flavoring is added to the oral liquid memantine formulation.

For administration by inhalation, memantine can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection can be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

Dosage units for rectal application can be solutions or suspensions or can be prepared in the form of suppositories or retention enemas comprising memantine in a mixture with a neutral fatty base, or gelatin rectal capsules comprising the active substances in admixture with vegetable oil or paraffin oil.

The formulations of the invention can be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient can be in powder form for reconstitution with a suitable vehicle, e.g., sterile pyrogen-free water, before use. For parental administration, the rate of infusion must be carefully controlled due to the relatively long half-life of memantine in the blood stream.

The invention also provides a pharmaceutical pack or kit comprising one or more containers containing memantine and, optionally, more of the ingredients of the formulation. In a specific embodiment, memantine is provided as an oral solution (2 mg/ml) for administration with the use of a 2 teaspoon capacity syringe (dosage KORC®). Each oral syringe has blue hatch marks for measurement, with lines on the right side of the syringe (tip down) representing tsp units, and those on the left representing ml units.

Dosages. Preferably, the optimal therapeutically effective amount should be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

Suitable daily doses of the active compounds of the invention in therapeutic treatment of humans are about 0.01-10 mg/kg bodyweight on peroral administration and 0.001-10 mg/kg bodyweight on parenteral administration.

For adults, suitable daily doses of memantine are within the range from about 5 mg to about 100 mg per day, preferably, from about 20 to about 40 mg per day.

For pediatric subjects aged 4-14, it is anticipated that memantine is administered as an oral, liquid dosage form, at about 0.5 mg/kg/day, up to a maximum dose of about 20 mg/day. Titration to the maximum dose over about 4 weeks from a lower initial starting dose, is highly recommended. For liquid, oral administration, memantine is dissolved in about one-half the liquid equivalent of the dose. For example, 12.5 mg memantine will be dissolved in 10 ml of the liquid formulation for administration.

Treatment duration can be short-term, e.g., several weeks (for example 8-14 weeks), or long-term until the attending physician deems further administration no longer is necessary.

Memantine may be administered as monotherapy, or in combination with another agent prescribed for the treatment of autistic spectrum disorders or combined type ADHD, with the exception of an agent that is a GABA analogue if the autistic spectrum disorder is Asperger syndrome.

### EXAMPLES

The invention is also described by means of particular examples. However, the use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### Open-Label Evaluation of Memantine for the Treatment of Autism

This clinical study will be conducted as an open-label, multi-center, dose-finding outpatient study assessing memantine in pediatric patients diagnosed with Autistic disorder (DSM-IV-TR criteria). Autistic disorder is characterized by impairment in social interaction, in communication skills, and by stereotyped/repetitive behaviors.

Patient population and diagnosis. The study population will consist of 20 children between the ages of 5 and 17 (preferably from between ages 6 and 12) who have been diagnosed with autistic behaviors, and who have not improved on other medications. Diagnosis is confirmed, or in the case of naïve patients, made, using the DSM-IV-TR™ criteria (described above) based on clinical evaluation and a semi-structured interview by a health professional, the Autistic Diagnostic Observation Schedule (ADOS-G). At Screening, patients must have a non-verbal IQ score of ≧40 as measured by the Test of Nonverbal Intelligence (TONI-3).

In addition, scales such as the CHAT, CARS, PIA, (GARS), Behavior BRIAC, ADI-R, ADOS-G, and DISCO, described above, may be used to diagnose autism or gauge severity of symptoms. Other measures may include the Communication and Symbolic Behavior Scale (CSBS) and the Ritvo Real-life Rating Scale. MRIs may also be performed to evaluate observed differences in total brain, parieto-temporal lobe, and cerebellar hemisphere volumes, or in sizes of the size of amygdala, hippocampus, and corpus callosum (Brambilla et al., Brain Res Bull. 2003; 61(6):557-69) in autism, or the mesenscephalon in Asperger's syndrome (Nieminen-von Wendt, Int. J. Circumpolar Health. 2002; 61 Suppl 2:22-35).

Patients will be excluded if they present with a primary psychiatric diagnosis of schizophrenia or bipolar disorder at Screening, with a history of major depressive disorder within the past 6 months, with a history of other neurological disease including but not limited to seizure/epilepsy. Patients also will be excluded if have initiated psychotherapy, behavior therapy, and/or cognitive therapy within 2 months prior to Screening, if are taking or have taken any excluded concomitant medications prior to the minimum allowable interval before Screening, and if they have received treatment with any investigational drug within 30 days or 5 half lives (whichever is longer) prior to study entry. Other exclusion criteria also apply.

Concomitant drugs which are not permitted during the study include anorexics, anticholinergics, anticoagulants, anticonvulsants, antidepressants, antihypertensives, anti-obesity drugs, antipsychotics, anxiolytics, antiviral agents, cholinesterase inhibitors, hormones and hormone suppressants, hypolipidemics, muscle relaxants, psychotropic drugs, sedatives or hypnotics, systemic steroids, systemic antifungal agents, chronic anti-diarrheal preparations, and stimulants and other ADHD treatments,

The “Screened Population” will consist of all patients who had a screening visit with an assigned screen number.

The “Safety Population” will consist of all patients who took at least one dose of study medication.

The “Intent-to-Treat (ITT) Population” will consist of all patients in the Safety Population with at least one post-baseline efficacy assessment of CGI-I.

Study design and treatment. Patients who meet eligibility criteria at the Screening (Week 2) will be started on 8 weeks of open-label treatment with memantine. This study will be conducted as a dose-finding study in which all patients, in the absence of dose-limiting adverse events, will be titrated from a starting dose of 2.4 mg/day at the start of Week 1 to 4.8 mg/day at the start of Week 2 to 7.2 mg/day at the start of Week 3 to a maximum dose of 10 mg/day at the start of Week 4. Patients will remain on the maximum tolerated daily dose (up to 10 mg/day) from the start of Week 5 to the end of Week 8. For patients experiencing dose-limiting adverse events, dose reduction to the next lowest tolerated dose with subsequent attempts to retitrate upward will be permitted. Patients who cannot tolerate the 2.4 mg/day dose will be dropped from the study and replaced.

The study will have a total of 11 visits: Screening (Visit 1), Baseline (Visit 2; Week 0), and Visits 3 to 10 (weekly visits at Weeks 1 through 8), and Visit 11 (1 to 5 days after the last dose of study drug) (see Schedule of Evaluations, Section 3.0). Screening may be completed as 2 separate evaluations, Part 1.1 and Part 1.2 within the 2-week screening period. If necessary, Visits 2 to 10 may be conducted up to 3 days before or after the final day of the study week. All visits after screening should be scheduled such that the Baseline visit is considered the 0 time point.

Patients who miss two consecutive visits may be discontinued from the study and replaced.

Drug dosing and administration. Memantine HCl will be supplied by Forest Laboratories. Medication is to be administered in the morning as a single daily dose.

The amount of study medication dispensed, titration, and maintenance schedule by visit and week are shown below.

| | End of: | | | | | | | |
|---|---|---|---|---|---|---|---|---|
| | Titration | | | | Maintenance | | | |
| | Visit 2 | Visit 3 | Visit 4 | Visit 5 | Visit 6 | Visit 7 | Visit 8 | Visit 9 |
| Dose | (Week 0) | (Week 1) | (Week 2) | (Week 3) | (Week 4) | (Week 5) | (Week 6) | (Week 7) |
| mg/day | 2.4 | 4.8 | 7.2 | 10 | 10 | 10 | 10 | 10 |

Only doses specified in the titration and maintenance schedule (i.e., 2.4 mg/day, 4.8 mg/day, 7.2 mg/day, and 10 mg/day) will be allowed. Patients unable to tolerate the minimum dose of 2.4 mg/day will be discontinued from the study.

Dose adjustments will be permitted for patients experiencing dose-limiting adverse events at the discretion of the investigator. The study physician must be made aware of any dosing modifications. Dose adjustment will always proceed from the intolerant dose to the next lowest or next highest allowed dose.

From the end of Week 2 (Visit 4) to the end of Week 5 (Visit 7), patients experiencing dose-limiting adverse events may have their daily dose of memantine decreased to the dose immediately below the intolerant dose. That is, patients who are unable to tolerate memantine 4.8 mg/day may have their dose reduced to 2.4 mg/day; patients unable to tolerate memantine 7.2 mg/day may have their dose reduced to 4.8 mg/day; and patients unable to tolerate memantine 10 mg/day may have their dose reduced to 7.2 mg/day. If a dose reduction occurs, an attempt may be made to retitrate the dose of memantine back to the higher (previously intolerant) dose in the next study week. Patients unable to tolerate this retitration will be allowed to continue in the study at the lower tolerated daily dose. Patients able to tolerate the retitration may continue to have their dose of memantine increased on a weekly basis until the end of Week 5 (Visit 7). The highest dose tolerated by the end of Week 5 (Visit 7) will be the dose that is maintained during the remaining 3 weeks of the treatment (Visits 8, 9, 10).

During Weeks 6 to 8, patients experiencing dose-limiting adverse events will be allowed to further decrease their daily dose to the dose immediately below the intolerant dose. Up-titration will not be permitted during this time period. For this study, the minimum daily dose will be 2.4 mg/day and the maximum daily dose will be 10 mg/day.

A schematic of the schedule of permissible dose adjustments is presented below.

| | End of Week | | | | | | | |
|---|---|---|---|---|---|---|---|---|
| | Titration | | | | Maintenance | | | |
| | Visit 2 | Visit 3 | Visit 4 | Visit 5 | Visit 6 | Visit 7 | Visit 8 | Visit 9 |
| | (Week 0) | (Week 1) | (Week 2) | (Week 3) | (Week 4) | (Week 5) | (Week 6) | (Week 7) |
| Maximum Daily Dose [mg] | 2.4 | 4.8 | 7.2 | 10 | 10 | 10 | 10 | 10 |
| Dose Reduction allowed? | No | Yes | Yes | Yes | Yes | Yes | Yes | Yes |
| Dose Increase after Reduction? | — | No | Yes | Yes | Yes | No | No | No |

Evaluation. The primary objective is to provide preliminary safety and tolerability evaluations of memantine in pediatric patients with autistic disorder.

The secondary objectives are to evaluate the pharmacokinetics of memantine and to provide preliminary evaluations of efficacy based on an 8-week administration of memantine. Efficacy will be evaluated based on the change from Baseline (Visit 2) to the end of Week 8 (Visit 10) in the CGI-I, CGI-S, RUPP-TSA, ABC-RCV, PPVT III, and MTS task. See above for descriptions of these scales. EEGs can also be used to detect differences in frontal lobe electrical activity.

Descriptive statistics (n, mean, standard deviation, median, minimum, and maximum) for the total score and the change from Baseline (Visit 2) at each post-baseline visit will be presented for each efficacy parameter.

The primary analysis will be based on observed data. Missing values will not be imputed for the purposes of presentation. A last observation carried forward (LOCF) analysis will also be presented for each efficacy assessment. Using this approach, the last observed value before a missing value at a post-baseline visit is carried forward to impute the missing value, provided that at least one post-baseline assessment is available.

Pharmacokinetics. The plasma concentration-time profile of memantine in pediatric patients with autistic disorder will be described using a mixed effects population pharmacokinetic model. Pharmacokinetic analyses will be carried out using NONMEM® in order to estimate the pharmacokinetic parameters of memantine.

Adverse events. Adverse event (AE) monitoring will be performed at all study visits except Screening, including early termination. An AE is any untoward medical occurrence in a patient or clinical investigation subject administered a pharmaceutical product. It is not necessary that the AE have a causal relationship to treatment with the product.

An AE therefore is any unfavorable and unintended sign (for example, a clinically significant abnormal laboratory finding) symptom, or disease temporally associated with the use of study medication, whether or not considered related to study medication. Adverse events include treatment-emergent adverse events, serious adverse events, adverse events leading to premature study discontinuation, and deaths. An AE occurring during the open-label treatment period will be counted as a treatment-emergent AE (TEAE) if it is not present prior to the first dose of study medication or if it is present prior to the first dose of study medication but increases in severity following the first dose of study medication.

The number (percentage) of patients with TEAEs will be tabulated by body system and preferred term. Within a specific category (i.e., a specific body system or preferred term), the patient is to be counted only once if the patient had more than one event reported. Listings will be provided for all patients with serious adverse events (SAEs) and adverse events leading to premature study discontinuation (ADOs). A listing of death(s) will also be provided, if applicable.

### Results

It is expected that memantine treatment will demonstrate significant improvements in secondary endpoints, i.e., improvements in the behavior and symptomology, compared to placebo-treated individuals. Such improvements maybe be in one or more of the following: improvements in eye contact, awareness of surroundings, verbal communication skills, response to verbal commands, tolerance of mild changes in routine, and sensory perception; and reductions in hyperactivity, aggression, self-injurious behavior, and repetitive behavior.

For example, improvements on the diagnostic scales, ADOS-G and TONI-3, which measure social and communicative behavior, and non-verbal intelligence, respectively, is expected. Similarly, improvements on the efficacy scales such as the CGI-I, CGI-S, RUPP-TAS, ABC-CV, PPVT III, and MTS scales, are expected with memantine compared to placebo.

### Example 2

### Randomized, Double-Blind, Placebo-Controlled Trial for Memantine for the Treatment of Autistic Spectrum Disorder

The primary objective of this study is to evaluate the efficacy and safety of memantine in pediatric patients with autism.

Design. This clinical study will be conducted as a multicenter, randomized, double-blind, placebo-controlled, parallel-group, flexible dose study comparing memantine to placebo in pediatric outpatients diagnosed with autism using the DSM-IV, Autism Diagnostic Observation Schedule (ADOS) and Autism Diagnostic Inventory-Revised (ADI-R) criteria. The study will consist of 2 weeks of single-blind, placebo lead-in treatment followed by 12 weeks of double-blind, flexible-dose treatment At the end of the single-blind period, patients meeting the entry criteria for this study will be randomized (1:1) to one of 2 double-blind treatment groups (memantine or placebo).

Patient population and diagnosis. The study will consist of patients from about 5 to about 12 years who meet diagnostic criteria for autism and who are either nto retarded or only mildly retarded.

No concomitant psychotropic medication or medication with a psychotropic component will be permitted.

Dosage. Memantine will be administered orally as a single daily dose of 4 capsules The proposed dosage regimen is as follows (although it will be subject to change): For the 12 week double-blind flexible dose treatment, the starting dose will be 3 mg/day for the first 2 weeks of double-blind treatment. In the absence of dose-limiting adverse events, memantine dosage will be titrated upward to 6 mg/day at Week 3, then up to 9 mg/day at Week 4, then up to 12 mg/day at Week 5, and then up to 18 mg/day for Weeks 6 through 12. All patients must reach a minimum dose of 6 mg/day by the end of Week 6 in order to be eligible to remain in the study. Flexible dose reduction is permitted before Week 8, and all treatments will remain fixed at the maximum tolerated dose for Weeks 8 through 12. Patients who complete the 12-week, double-blind phase will be eligible to enroll in an open-label extension.

Evaluation. Primary efficacy parameters used to evaluate the patients will be the CGI-S (Clinical Global Impression-Severitiy) and CGI-I (Clinical Global Impression-Improvement). The CGI subscale of severity (CGI-S) is assessed at baseline whereas the CGI-Improvement (CGI-I) scale is sensitive to measuring change from the baseline severity rating at each visit or at certain designated visits during the study and at end point. The ratings for CGI-S range from “1” (not ill) to “7” (extremely ill); the ratings for CGI-I, range from “1” (very much improved) to “7” (very much worsened). CGI-S will be performed at Screening (Visit 1) to determine the patient's fulfillment of the inclusion criteria of a CGI-S score≧4 (moderately ill).

Secondary efficacy parameter used to evaluate the patients will be the Autism Diagnostic Observation Schedule-Generic (ADOS).

The secondary efficacy parameter will be mean change from Baseline at Week 12 in the total raw score of the items included in the Language and Communication (9 items) as well as the Reciprocal Social Interactions (10 items)

Statistical analysis. The primary efficacy parameter is the percentage of responders (defined as a score of 1 or 2) on the CGI-I rating scale at Week 12 using the last observation carried forward (LOCF) approach. The primary analysis will be based on Cochran-Mantel-Haenszel (CMH), controlling for study center, for testing the null hypothesis of no difference in CGI-I positive response rate between the two treatment groups. Safety measures are based on the Safety Population, defined as all randomized patients who receive at least one dose of double-blind study drug. Efficacy measures are based on the Intent-to-Treat (ITT) Population, defined as all patients in the Safety Population with at least one post-Baseline assessment of CGI-I

### Results

It is anticipated that memantine will be well-tolerated and will be shown to be efficacious for the treatment of autism compared with placebo within the administered dosage range of between about 6-18 mg/day.

### Example 3

### Open-Label Evaluation of Memantine for the Treatment of ADHD

The primary objective of this study was to provide preliminary safety and tolerability evaluations of memantine in pediatric patients with ADHD combined type. The secondary objectives of the study were to evaluate the pharmacokinetics of memantine in this patient population, and to provide preliminary evaluations of efficacy on effect based on a 8-week administration of memantine.

Design. This clinical trial was conducted as an open-label, single center, dose-finding outpatient study assessing memantine in pediatric patients diagnosed with ADHD combined type (DSM-IV-TR™ criteria). ADHD combined type is characterized by the presence of significant inattentive and hyperactive/impulsive symptomatology.

This study involved a total of ten clinic visits: Screening, Baseline, weekly visits at the end of Weeks 1 to 8, and a Final visit 1 to 5 days following the last dose of study drug. The maximum duration between the completion of the Screening and the Baseline visits was 2 weeks. In order to reduce patient/parent burden the Screening visit could have been completed in 2 separate evaluations, part 1.1 and part 1.2 within the 2-week period.

Patient Population. The study enrolled sixteen (16) male or female outpatients between the ages of 6 and 12 years, mean age of 8 years, diagnosed with ADHD combined type by DSM-IV-TR™ criteria based on clinical evaluation and a semi-structured interview, the Schedule for Affective Disorders and Schizophrenia for School-Age Pediatric patients-Present and Lifetime (K-SADS-PL). At baseline, patients must have had a total score on the Attention-Deficit/Hyperactivity Disorder Rating Scale-IV (ADHD-IV-RS) Parent Version of 24 or greater, a Clinical Global Impression-ADHD-Severity (CGI-ADHD-S) score≧4, and verbal intelligence in the non-mentally retarded range as measured by a standardized score of ≧70 on the Peabody Picture Vocabulary Test, Third Edition (PPVT III).

A washout period was allowed for candidates on psychoactive medication (except depot neuroleptic and narcotics) during the Screening period for up to 3 weeks or 5 half lives, whichever was shorter. For patients on psychoactive medication, only those with an unsatisfactory therapeutic response were eligible for the study.

Patients presenting with any primary psychiatric diagnosis other than ADHD combined type (oppositional defiant disorder is allowed) at Screening, or with a history of neurological disease including but not limited to seizure/epilepsy (except simple febrile seizures), movement disorder, and Tourette's Disease were excluded. Also excluded, except as indicated in the foregoing paragraph, were patients who have been treated with any stimulant drug [including methylphenidate (Ritalin®, Concerta®), atomoxetine (Strattera®), amphetamine mixed salt (Adderall®), or pemoline (Cylert®)], antidepressant or anxiolytic medication, neuroleptic medication, or epileptic medication within 1-2 weeks of the Screening Visit.

Concomitant CNS-acting medications including but not limited to anorexics, anticholinergics, anticonvulsants, antidepressants, antipsychotics, antiobesity agents, chronic antiemetics, cholinesterase inhibitors, hormone and hormone suppressants, sedatives or hypnotics, or stimulants were not be permitted for inclusion in this study. Other concomitant medications excluded include anesthetics, anticoagulants, chronic antidiarrheal agents, systemic antifungal agents, antihypertensives, chronic anti-inflammatory drugs, antineoplastics, antiviral agents, hypolilpidemics, muscle relaxants, systemic steroids, and chronic vaccines.

Dosages. Sixteen (16) outpatients with a diagnosis of ADHD combined type received treatment with memantine for a total of 8 weeks. Patients were enrolled in to two groups of 8 patients each, Cohort 1 and Cohort 2. Dose adjustments were permitted for patients experiencing dose-limiting adverse events. For Cohort 1 patients, treatment consisted of a 4 week titration period from a starting dose of 2.4 mg/day at the start of week 0, to 4.8 mg/day at the start of week 1, to 7.2 mg/day at the start of Week 2 to a maximum dose of 10 mg/day at the start of week 3, with a maintenance dose of 10 mg/day continuing to the end of Week 8. For Cohort 2 patients, treatment consisted of a 4-week titration period from a starting dose of 4.8 mg/day at the start of Week 0, to 10 mg/day at the start of Week 1, to 14.8 mg/day at the start of Week 2, to a maximum dose of 20 mg/day at the start of Week 3, with a maintenance dose of 20 mg/day continuing to the end of Week 8.

The amount of study medication dispensed, titration, and maintenance schedule by visit and week for Cohort 1 and Cohort 2 are shown in Table 1 below.

TABLE 1
Dosing Titration and Scheduling

| | Titration | | | | Maintenance | | | |
|---|---|---|---|---|---|---|---|---|
| | Visit 2 | Visit 3 | Visit 4 | Visit 5 | Visit 6 | Visit 7 | Visit 8 | Visit 9 |
| | End of | End of | End of | End of | End of | End o | End of | End of |
| | Week 0 | Week 1 | Week 2 | Week 3 | Week 4 | Week 5 | Week 6 | Week 7 |
| Cohort 1 | 2.4 | 4.8 | 7.2 | 10 | 10 | 10 | 10 | 10 |
| Cohort 2 | 4.8 | 10 | 14.8 | 20 | 20 | 20 | 20 | 20 |

A schematic of the schedule of permissible dose adjustments is presented in Table 2 below.

TABLE 2
Dosing Titration and Scheduling Adjustments

| | Titration | | | | Maintenance | | | |
|---|---|---|---|---|---|---|---|---|
| | Visit 2 | Visit 3 | Visit 4 | Visit 5 | Visit 6 | Visit 7 | Visit 8 | Visit 9 |
| | End of | End of | End of | End of | End of | End o | End of | End of |
| | Week 0 | Week 1 | Week 2 | Week 3 | Week 4 | Week 5 | Week 6 | Week 7 |
| Cohort 1 | 2.4 | 4.8 | 7.2 | 10 | 10 | 10 | 10 | 10 |
| Max daily dose | | | | | | | | |
| Cohort 1 | No | Yes | Yes | Yes | Yes | Yes | Yes | Yes |
| Dose Reduction allowed? | | | | | | | | |
| Cohort 1 | — | No | Yes | Yes | Yes | No | No | No |
| Dose Increase after Reduction? | | | | | | | | |
| Cohort 2 | 4.8 | 10 | 14.8 | 20 | 20 | 20 | 20 | 20 |
| Max daily dose | | | | | | | | |
| Cohort 2 | No | Yes | Yes | Yes | Yes | Yes | Yes | Yes |
| Dose Reduction allowed? | | | | | | | | |
| Cohort 2 | — | No | Yes | Yes | Yes | No | No | No |
| Dose Increase after Reduction? | | | | | | | | |

Evaluation and assessment. The study had a total of 10 visits: Screening (Visit 1), Baseline (Visit 2; week 0), and Visits 3 to 9 (weekly visits at end of weeks 1, 2, 3, 4, 5, 6, 7, and 8). The Screening Visit may have been completed as 2 separate evaluations, part 1.1 and part 1.2 within the 2-week screening period. If necessary, study Visits 2 to 10 were conducted up to 3 days before or after the final day of the study week. All visits after the screening visit were scheduled such that the Baseline visit is considered the 0 time point.

Patients who missed 2 consecutive visits were discontinued from the study. The “Screened Population” consisted of all patients who had a screening visit with an assigned screen number. The “Safety Population” consisted of all patients who took at least one dose of study medication. The “Intent-to-Treat (ITT) Population” consisted of all patients in the safety population with at least one post baseline efficacy assessment of ADHD-IV-RS or CGI-ADHD-S. ADHD-IV-RS or CGI-ADHD-S were administered 4 times per patient, once at Screening, once at baseline, once at the end of Week 4 and once at the end of Week 8. Patients who did not tolerate the required daily dose during Week 0 of dosing were discontinued from the study with replacement. Those patients unable to tolerate the minimum daily dose after the Week 1 of dosing were discontinued from the study without replacement.

Efficacy parameters used for evaluation were the ADHD-IV-RS, CGI-ADHD-S, CCPT-II, Woodcock Johnson III reading and math fluency tests, and the Stroop test, described above.

The primary analysis for each efficacy parameter was based on observed cases. Only patients completing the evaluation were included in the OC analysis, missing data were not imputed. A last observation carried forward (LOCF) analysis was also performed for each efficacy assessment. Using this approach, the last observed value before a missing value at a post baseline visit is carried forward to impute the missing value, provided that at least one post baseline assessment is available. All patients in Cohort 1 and Cohort 2 had at least one post-baseline visit and are included in the LOCF analysis.

Pharmacokinetic evaluation. Plasma samples were to be obtained at Weeks 1 (Visit 3), 2 (Visit 4), 3 (Visit 5), 4 (Visit 6), and 5 (Visit 7) or early termination at random times post-dosing. For Weeks 1, 2, 3, and 4, PK sampling was to be collected during the following time windows: trough (0 hour), >0-2 h, >2-4 h and 4-8 h; on Week 5, PK sampling could take place at any time between >0-8 hours. Prior to obtaining the trough blood draw, the patient should not have taken the morning dose of study medication until after arriving at the research site for a morning visit and the trough sample is drawn.

Pharmacokinetic analyses were carried out using NONMEM® in order to estimate the pharmacokinetic parameters of memantine.

Adverse events. Adverse event (AE) monitoring was performed at all study visits except Screening, including early termination. An AE is any untoward medical occurrence in a patient or clinical investigation subject administered a pharmaceutical product. It is not necessary that the AE have a causal relationship to treatment with the product.

An AE therefore is any unfavorable and unintended sign (for example, a clinically significant abnormal laboratory finding) symptom, or disease temporally associated with the use of study medication, whether or not considered related to study medication. Adverse events include treatment-emergent adverse events, serious adverse events, adverse events leading to premature study discontinuation, and deaths. An AE occurring during the open-label treatment period is counted as a treatment-emergent AE (TEAE) if it is not present prior to the first dose of study medication or if it is present prior to the first dose of study medication but increases in severity following the first dose of study medication.

The number (percentage) of patients with TEAEs were tabulated by body system and preferred term. Within a specific category (i.e., a specific body system or preferred term), the patient was counted only once if the patient had more than one event reported. Listings were provided for all patients with serious adverse events (SAEs) and adverse events leading to premature study discontinuation (ADOs). A listing of death(s) were to be provided, if applicable.

### Results

Memantine demonstrated improvements in the behavior and symptomology of combined type ADHD for patients in Cohort 2, thereby being an effective treatment for combined type ADHD. Since memantine is not a stimulant, as are conventional ADHD medications which are designated controlled substances, there is no anticipated potential for drug abuse.

Patient Disposition. All 8 patients in Cohort 1 discontinued prior to week 8; 7 for insufficient therapeutic efficacy, 5 of whom discontinued within Weeks 1-4, and 1 withdrew consent, In Cohort 2, 4 patients completed the study, 3 discontinued for insufficient therapeutic efficacy, 1 of whom discontinued within Weeks 1-4, and 1 was lost to follow up, All 16 patients completed at least one post-baseline efficacy assessment and were included in the ITT population.

The mean duration of treatment was 29 days in Cohort 1, with a mean daily dose of 5.6 mg/day. The mean duration of treatment was 42.25 days in Cohort 2, with a mean daily dose of 13.5 mg/day. With the exception of 2 patients in Cohort 2 (medication bottles were not returned), all patients were over 75% compliant.

### Efficacy Parameters

ADHD-IV-RS. Efficacy was evaluated using the ADHD-IV-RS. By visit scores and change from baseline at Week 8 for cohorts 1 and 2 based on the OC and LOCF analyses are presented in Table 3 and Table 4 below, respectively. At the end of 8 weeks the patients receiving memantine in cohort 2 showed improvement on the ADHD-IV-RS in the OC analysis.

TABLE 3
Change from Baseline in ADHD-IV-RS Parameters: Mean ± SD ITT Population, OC

| Parameter | Cohort | Baseline | Week 4 | Change from Baseline | Week 8 | Change from Baseline |
|---|---|---|---|---|---|---|
| ADHD-IV-RS | 1 | 44.6 ± 7.50 | 42.8 ± 8.33 | −3.5 ± 2.38 | — | — |
| | 2 | 41.9 ± 4.64 | 29.5 ± 13.32 | −11.5 ± 9.65 | 22.3 ± 8.66 | −16.5 ± 8.19 |

TABLE 4
Change from Baseline to Week 4 and Week 8 in ADHD-IV-RS:
Mean ± SD ITT Population, LOCF

| Parameter | Cohort | Baseline | Week 4 | Change | Week 8 | Change |
|---|---|---|---|---|---|---|
| ADHD-IV-RS | 1 | 44.6 ± 7.50 | 42.8 ± 8.33 | −3.3 1.97 | 40.9 ± 8.53 | −3.8 ± 2.31 |
| | 2 | 41.9 ± 7.50 | 31.3 ± 13.05 | −10.9 8.97 | 32.9 ± 12.76 | −9 ± 10.00 |

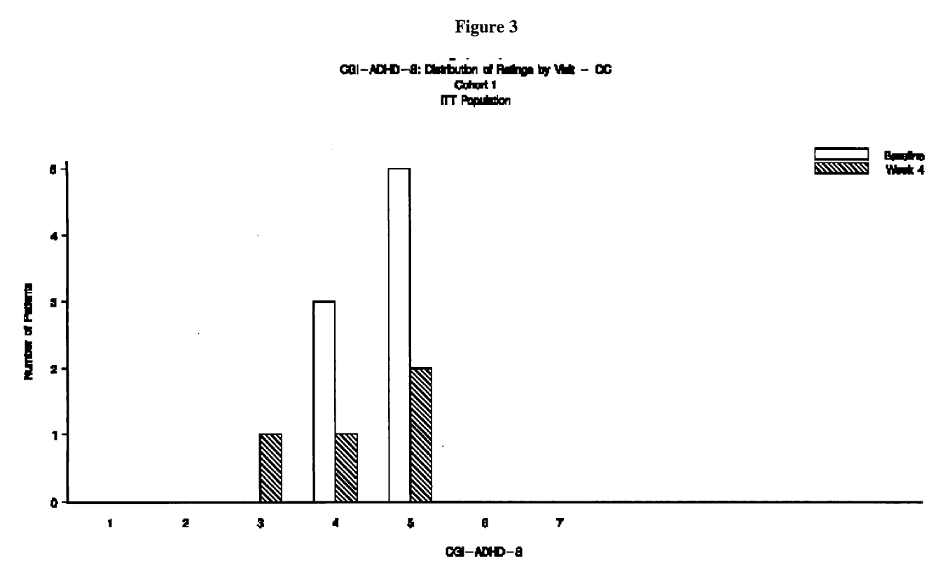

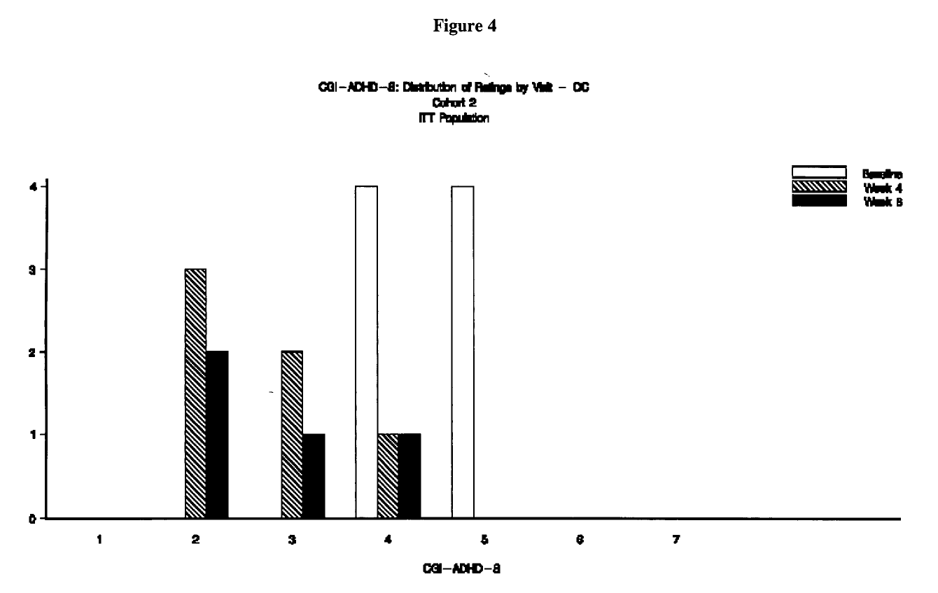

CGI-ADHD-S. In an OC analysis of Cohort 2 (for patients who completed the evaluation), 75% of the patients were considered borderline or markedly ill compared to 100% being considered moderately or markedly ill at baseline (as indicated by a baseline score of 4-5 on the vertical axis of FIGS. 3 and 4). At the end of 8 weeks, the distribution of scores in an OC analysis for cohort 1 and cohort 2 at baseline, Week 4 and Week 8 are presented in FIGS. 3 and 4.

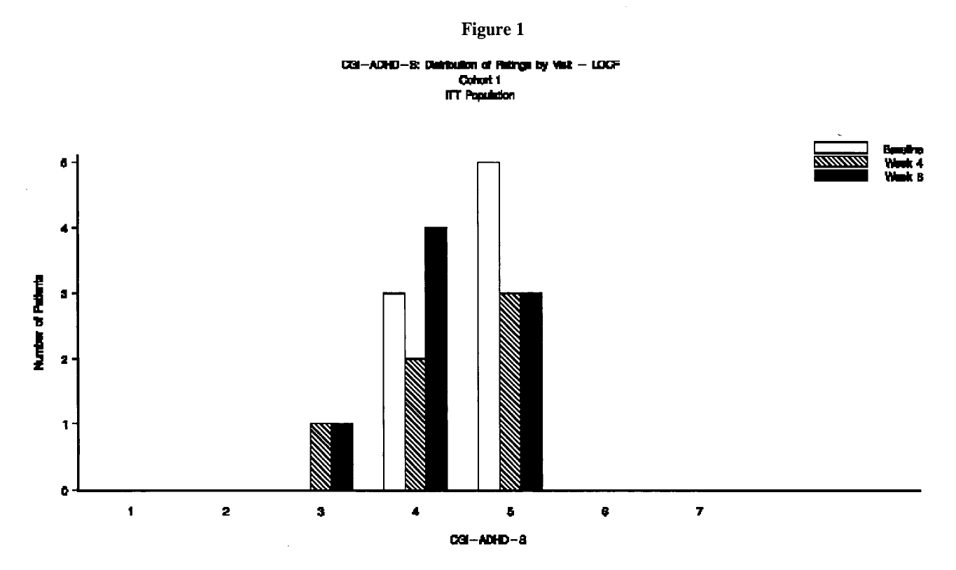

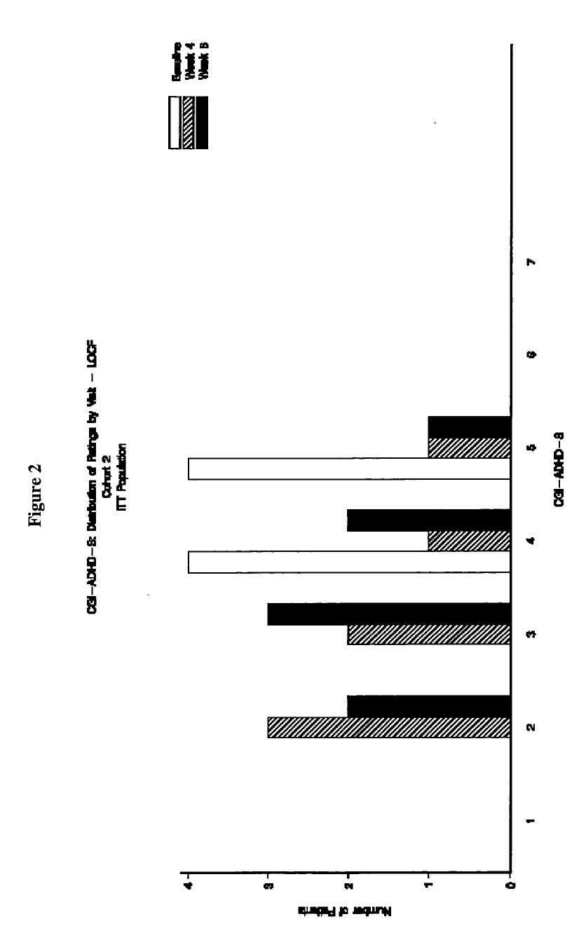

The distribution of scores in an LOCF analysis for Cohort 1 and Cohort 2 at baseline, Week 4 and Week 8 are presented in FIGS. 1 and 2. As indicated above, all of the patients dropped out prior to completing the study for lack of efficacy in Cohort 1. However, as all completed at least one post-baseline assessment, they are included in the LOCF analysis.

Similarly, the LOCF analysis for Cohort 2 includes the 4 patients who did not complete the study as they each had at least one post-baseline assessment. In addition to the 4 patients who completed the study in Cohort 2, two additional patients reached the maximum daily dose of 20 mg/day.

### CONCLUSION

Memantine was well tolerated in patients with ADHD. In addition, after 8 weeks of treatment, improvement in two efficacy parameters was observed, indicating that memantine was of benefit in the treatment of ADHD in the 25% of patients completing the study.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method for the treatment of an individual diagnosed with a childhood behavioral disorder wherein the disorder is not Rett syndrome, comprising administering to a subject in need thereof a therapeutically effective amount of memantine.

2. The method of claim 1, wherein the disorder is autistic spectrum disorder.

3. The method of claim 2, wherein the autistic spectrum disorder is selected from the group consisting of autism, Asperger syndrome, pervasive development disorder, and childhood disintegrative disorder.

4. The method of claim 3, wherein the autistic spectrum disorder is autism.

5. The method of claim 1, wherein the disorder is combined type Attention-Deficit/Hyperactivity Disorder.

6. The method of claim 1, wherein the subject is between the ages of 5 and 17.

7. The method of claim 1, wherein the subject is between the ages of 6 and 12.

8. The method of claim 2 wherein the subject is an adult.

9. The method of claim 5, wherein the subject is an adult.

10. The method of claim 1, wherein the memantine is administered in a dosage range from about 1.25 mg to about 100 mg/day.

11. The method of claim 10, wherein the memantine is administered in a dosage range from about at about 5 to about 40 mg/day.

12. The method of claim 10, wherein the memantine is administered at a dose of about 10-20 mg/day.

13. The method of claims 1, wherein the memantine is administered once a day or twice a day (b.i.d.).

14. The method of claim 1, wherein the memantine is administered once a day in a modified release formulation.

15. The method of claim 1, wherein the memantine is administered in a flavored, oral, liquid formulation

16. The method of claim 1, wherein the treatment comprises improvements in one or more of eye contact, awareness of surroundings, verbal communication skills, response to verbal commands, inattentiveness, hyperactivity, impulsivity, aggression, self-injurious behavior, tolerance of mild changes in routine, repetitive behavior, or sensory perception.

17. The method of claim 1, wherein the disorder is selected from the group consisting of an anxiety disorder, bipolar disorder, depression, disruptive behavior disorder, dyslexia, fragile X syndrome, learning disabilities, obsessive-compulsive disorder (OCD), oppositional defiant disorder, reactive attachment disorder, separation anxiety disorder and Tourette's syndrome.

18. The method of claim 1, wherein memantine is administered in combination with another agent prescribed for the treatment of autistic spectrum disorders or combined type ADHD, with the exception of an agent that is a GABA analogue if the autistic spectrum disorder is Asperger syndrome.
